(19) United States
(12) Patent Application Publication
(10) Pub. No.: US 2002/0007150 A1
(43) Pub. Date: Jan. 17, 2002

(54) INSTRUMENT SHEATH AND BLADE GUIDE

(76) Inventor: STEPHEN H. JOHNSON, NEWPORT BEACH, CA (US)

Correspondence Address: BEN J. YORKS, IRELL & MANELLA LLP., 840 NEWPORT CENTER DRIVE, SUITE 400, NEWPORT BEACH, CA, 92660, US

(21) Appl. No.: 09/289,782

(22) Filed: Apr. 9, 1999

(51) Int. Cl. A61M 35/00; A61M 5/178

(52) U.S. Cl. 604/164.02; 604/294

(57) ABSTRACT

An instrument sheath that can be inserted into an incision formed in tissue. The sheath may protect the tissue from mechanical, thermal and other forms of trauma. The application also includes a blade guide that can be used to form an incision.

### BACKGROUND OF THE INVENTION

1. Field of the Invention

The present invention relates to a sheath that can be inserted into an incision of tissue and provides mechanical and thermal protection to the tissue from an instrument that passes through the sheath. The present invention also relates to a blade guide that guides a blade to form an incision.

2. Background Information

There has been developed a medical procedure commonly referred to as phacoemulsification (“phaco”) that is performed to remove a cataractous lens. A phaco procedure includes the steps of making an incision in the cornea and inserting a tip that is manipulated by a surgeon to break and remove the lens. The tip is typically driven by an ultrasonic device which imparts a vibratory energy to the lens. The tip extends from a handpiece that is coupled to an irrigation line and an aspiration system. The irrigation line provides an irrigation fluid to the anterior chamber of the cornea. The aspiration system pulls the irrigation fluid and emulsified lens from the cornea.

FIG. 1 shows a typical surgical instrument 1 used to perform a phaco procedure. The surgical instrument 1 includes an outer irrigation sleeve 2 that is attached to an outer case 3 of the instrument 1. An ultrasonically driven tip 4 extends from the case 3 and through the sleeve 2. The vibrating tip 4 is used to emulsify the lens of a cornea 5.

The case 3 has an irrigation inlet port 6 that is connected to an irrigation line (not shown). The irrigation line provides an irrigation fluid to the instrument. The outer sleeve 2 is separated from the tip 4 to create a channel 7 that allows irrigation fluid to flow from the inlet port 6, through an outlet port (not shown) in the sleeve 2 and into the cornea. The tip 4 has an aspiration channel 8 that is connected to an aspiration system. The irrigation fluid and emulsified lens are drawn through the channel 8.

The irrigation fluid must be delivered at a sufficient pressure and flowrate to maintain the intraocular pressure of the eye without damaging ocular tissue. It is desirable to provide an incision opening that is large enough to allow the tip 4 and sleeve 2 to be inserted into the cornea without allowing excessive irrigation fluid to leak back out of the anterior chamber.

The corneal tissue about the incision may exert a pressure that creates frictional contact between the inner surface of the sleeve and the oscillating tip. The frictional contact between the sleeve and the tip creates heat. The heat may burn the tissue of the cornea. Damage to the corneal tissue is irreversible and may result in a permanent impairment of the patient's vision. It is therefore important to avoid corneal burning during a phaco procedure. In some procedures that incision is larger than the instrument to allow fluid leakage that cools the tissue.

U.S. Pat. No. 5,354,265 issued to Mackool discloses an ultrasonic handpiece which has a flexible outer sleeve and a hard inner sleeve. The hard inner sleeve may reduce the amount of contact between the outer sleeve and the vibrating tip. The reduction in contact decreases the heat and probability of corneal burning. U.S. Pat. No. 5,282,786 issued to Ureche and U.S. Pat. No. 5,807,310 issued to Hood disclose irrigation sleeves that have bands which are constructed from a material such as TEFLON. The TEFLON sleeves have a stiffness that is greater than the stiffness of the rubber material. The TEFLON outer band may reduce the friction and corresponding heat generated between the cornea and the irrigation sleeve.

Having to provide a sleeve or band increases the complexity of the handpiece. Additionally, it has been found that even with a sleeve, relative movement between the tip and the cornea may stretch and tear corneal tissue at the incision. It would therefore be desirable to provide an apparatus and technique that can eliminate the disadvantages listed above without significantly increasing the cost or complexity of the instrument, or the procedure.

### SUMMARY OF THE INVENTION

The present application includes an instrument sheath that can be inserted into an incision formed in tissue. The sheath may protect the tissue from mechanical, thermal and other forms of trauma. The application also includes a blade guide that can be used to form an incision.

### BRIEF DESCRIPTION OF THE DRAWINGS

FIG. 1 is a cross-sectional view of an ultrasonic tip of the prior art;

FIG. 2 is a perspective view of an embodiment of a sheath of the present invention;

FIG. 3 is side sectional view showing the sheath inserted into an incision of a cornea and an instrument inserted through the sheath;

FIG. 4 is a perspective view of an embodiment of a blade guide;

FIG. 5 is a perspective view of a blade;

FIG. 6 is a top view showing the blade being moved across the blade guide and into a cornea.

### DETAILED DESCRIPTION

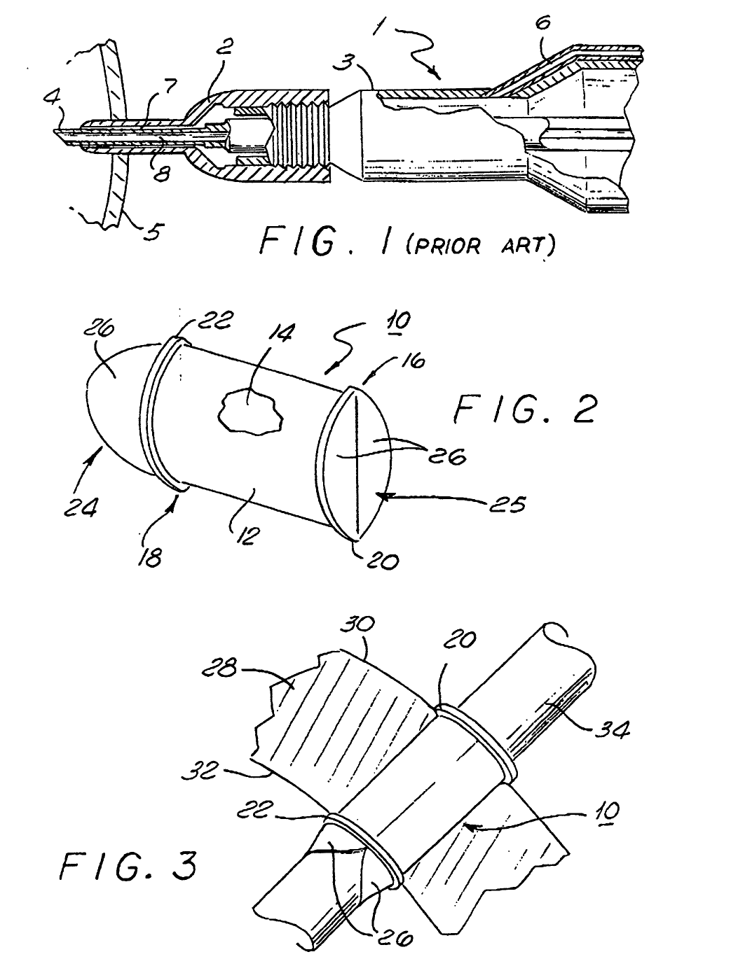

Referring to the drawings more particularly by reference numbers, FIG. 2 shows an embodiment of a corneal sheath 10 of the present invention. The corneal sheath 10 may include a shank 12 which has an inner channel 14 that extends from a proximal end 16 to a distal end 18 of the sheath 10. The proximal end 16 of the shank 12 may have a first annular rim 20. The distal end 18 of the shank 12 may have a second annular rim 22.

The sheath 10 may include a distal seal 24 that is located at the distal end 18 of the sheath 10 and a proximal seal 25 located at the proximal end 16 of the sheath 10. Each seal 24 and 25 may include a pair of lips 26 that can be deflected in an outward direction. The shank 12, rims 20 and 22, and seals 24 and 25 can all be constructed as an integrally molded piece. By way of example, the sheath 10 may be constructed from a silicon rubber or a molded plastic material.

As shown in FIG. 3, the sheath 10 can be inserted into an incision in a cornea 28. The sheath 10 can be pushed through the incision until the first annular rim 20 engages an outer surface 30 of the cornea 28. The first rim 20 provides a stop function which limits the penetration of the sheath 10. The second rim 22 may be flush with an inner surface 32 of the cornea 28. The first 20 and second 22 rims may be pressed against the inner 32 and outer 30 corneal surfaces to provide a double seal which prevents intraocular fluid from escaping the anterior chamber of the cornea 22. The second rim 22 may have a diameter that is smaller than the first rim 20 so that the sheath 10 can be removed from the incision of the cornea 28.

After the sheath 10 is inserted into the incision of the cornea 28 an instrument 34 can be inserted through the inner channel 14 of the sheath 10. The instrument 32 may be the tip of an ultrasonic handpiece that is used to emulsify a lens. Insertion of the instrument 34 deflects the lips 26 and opens the seals 24 and 25. The lips 26 may conform to the outer surface of the instrument to prevent fluid from leaking back through the sheath 10. The sheath 10 may be flexible enough so that the lips 26 move back to the original position and close the seals 24 and 25 when the instrument 34 is pulled out of the cornea 28.

The sheath 10 provides a protective element which prevents heat from being transferred between the ultrasonic instrument 34 and the corneal tissue. The instrument 10 does not require an outer protective sleeve as typically found in the prior art. This reduces the complexity and cost of producing and using the ultrasonic handpiece. Additionally, the sheath 10 structurally reinforces the cornea so that movement of the instrument does not stretch and tear the corneal tissue. The sheath 10 also allows the incision to be placed more posteriorly without fluid causing ballooning of conjunctiva of Tenon's capsule. Furthermore, the incision does not have to allow leakage to cool the tissue during surgery. The smaller incision and lack of leakage maintains the depth of the anterior chamber.

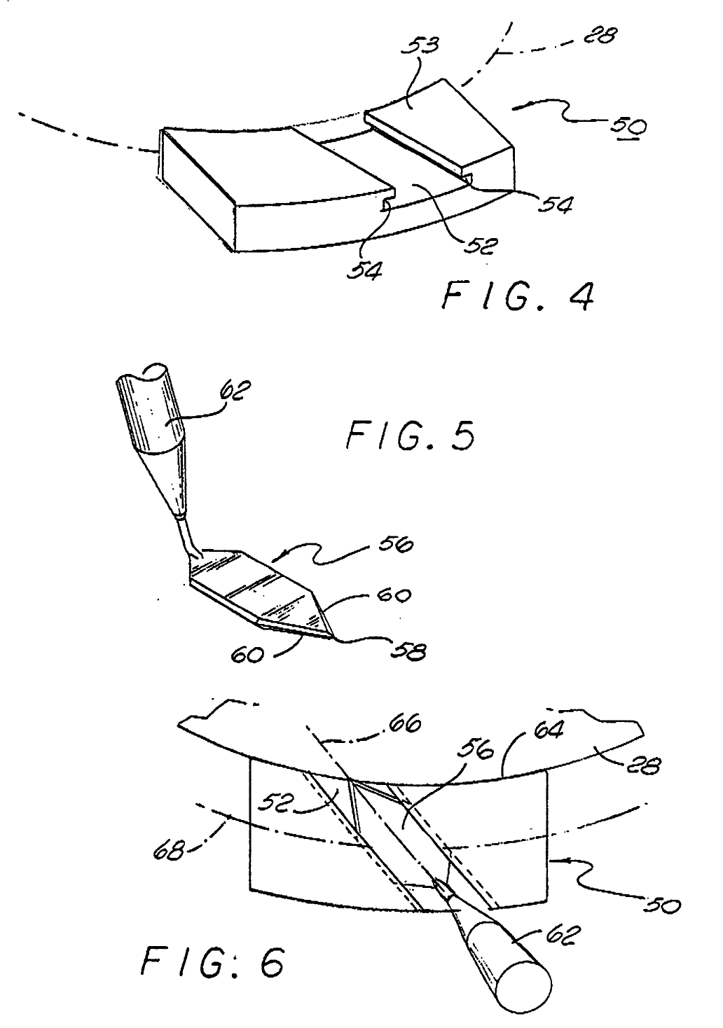

FIG. 4 shows an embodiment of a blade guide 50 that can be used to form an incision in a cornea. The blade guide 50 may include a groove 52 that extends across a width of a base plate 53. The groove 52 may have a T-shaped cross-section which has a pair of opposing channels 54.

The groove 52 may receive a blade 56 shown in FIG. 5. The blade 56 may have a sharpened tip 58 and edges 60 that can cut tissue. The blade 56 may extend from a handle 62 that can be held by a surgeon. The blade width and thickness may be such that the blade 56 can slide along the groove 52 of the blade guide 50 while being captured by the channels 54. The channels 54 allow lateral movement across the blade guide 50 without vertical movement out of the groove 52.

FIG. 6 shows the blade 56 moving across the blade guide 50 and into a cornea 28. The blade guide 50 may have an edge surface 64 that conforms to the outer surface of the cornea 28. The longitudinal axis 66 of the groove 52 may be at an oblique angle relative to the longitudinal axis 68 of the blade guide 50. This configuration allows the groove 52 to guide the blade 56 into the cornea at an oblique angle. The angular (tangential) incision increases the surface area of the corneal tissue incision permitting better self sealing. The blade guide 50 also insures a consistent cutting angle for each incision in every surgical procedure.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications may occur to those ordinarily skilled in the art. For example, although the blade guide 50 as described as being used in conjunction with the sheath 10, it is to be understood that the blade guide 50 can be used independent of the sheath 10 and vice versa. For example, the blade guide 50 can be used to form a second tangential incision that would be self sealing, through which an irrigation maintaining device could be placed during surgery.

What is claimed is:

1. A sheath that can be inserted into an incision of a cornea and receive an instrument, comprising:
a shank which is adapted to be inserted into the incision of the cornea, said shank having an inner channel that can receive the instrument.

2. The sheath of claim 1, further comprising a seal that is adapted to be opened when the instrument is inserted through said inner channel of said shank.

3. The sheath of claim 2, wherein said seal includes a pair of flexible lips adapted to be deflected when the instrument is inserted through said inner channel of said shank.

4. The sheath of claim 1, further comprising a first rim that is located at a proximal end of said shank.

5. The sheath of claim 4, further comprising a second rim that is located at a distal end of said shank.

6. A method for inserting an instrument through an incision in a cornea, comprising:
forming an incision in the cornea;
inserting a sheath into the incision; and,
inserting the instrument through the sheath.

7. The method of claim 6, wherein the sheath is inserted until a rim of the sheath engages the cornea.

8. A blade guide that is adapted to guide a blade into a cornea, comprising:
a base plate which has a groove that is adapted to receive and guide the blade.

9. The blade guide of claim 8, wherein said groove has a T-shaped cross-section.

10. The blade guide of claim 8, wherein said groove has a longitudinal axis that is at an oblique angle relative to a longitudinal axis of said base plate.

11. A method for forming an incision in a cornea, comprising:
placing a blade guide adjacent to the cornea, wherein the blade guide has a groove; and,
moving a blade across the groove and into the cornea.
